(19) United States
(12) Patent Application Publication
(10) Pub. No.: US 2021/0369668 A1
(43) Pub. Date: Dec. 2, 2021

(54) COMPOSITION EXHIBITING ENHANCED OXIDATIVE STABILITY

(71) Applicant: DSM IP ASSETS B.V., Heerlen (NL)

(72) Inventors: Weerasinghe M. INDRASENA, Halifax, NS (CA); Jaroslav A. KRALOVEC, Halifax, NS (CA); Bernd MUSSLER, Lahr (DE)

(21) Appl. No.: 17/286,926

(22) PCT Filed: Oct. 18, 2019
(86) PCT No.: PCT/EP2019/078328

§ 371 Date: Apr. 20, 2021

(30) Foreign Application Priority Data
Oct. 22, 2018 (EP) 18201657.6

(51) Int. Cl. A61K 31/355; A61K 31/202; A61K 31/375; A61K 9/48

(52) U.S. Cl. CPC A61K 31/355; A61K 9/48; A61K 31/375; A61K 31/202

(57) ABSTRACT

A composition comprising α-tocopherol acetate (also referred to as Vitamin E acetate “Vit E Acetate”) and docosahexaenoic acid ethyl ester (“DHA EE”), exhibits surprising oxidative stability.

### BRIEF DESCRIPTION OF THE INVENTION

This invention is a composition comprising α-tocopherol acetate (also referred to as Vitamin E acetate “Vit E Acetate”) and docosahexaenoic acid ethyl ester (“DHA EE”), which exhibits surprising oxidative stability.

### BACKGROUND OF THE INVENTION

DHA is a long chain fatty acid, and is a structural component of the brain, skin, and retina. It is commonly sold as a nutritional supplement, and can be obtained from fish oil, algal oil, and human breast milk.

There are two commonly marketed forms of DHA: the triglyceride form and the ethyl ester form. The ethyl ester form is a synthetic form, and is usually the one present in commercially available capsules containing concentrates of DHA. There is debate about which form is the more bioavailable one. The bioactivity of the two seems similar overall, although some studies show that the triglyceride form may be somewhat more beneficial.

Vitamin E is the common name applied to the eight naturally occurring lipid soluble plant-derived isomers and synthetic all racemic (all-rac)-α-tocopherol isomer mixtures. Vitamin E isoforms are not bioequivalent. Only α-tocopherol from naturally occurring RRR-α-tocopherol and other synthetic 2R-stereoisomeric forms of vitamin E commonly used in supplements are present in human circulation.

The use of a combination of one form of DHA and a type of Vitamin E administered simultaneously has been described in the art. For examples WO16/096685 (DSM IP ASSETS, B.V.) describes the use of the combination of DHA and Vitamin E for treatment of non-alcoholic fatty liver disease. Sadeghi et al 2018 describes the combination for dysmenorrhea (Gynecological Endocrinology Pages Ahead of PrintDOI:10.1080/09513590.2018.1450377); and WO2016/095778 (DSM IP ASSETS, B.V.) describes a combination for use in ameliorating diseases associated with particulate air pollution. None of these disclose the combination of Vitamin E acetate and DHA ethyl ester.

Like all oils, DHA EE is susceptible to oxidation, and can turn rancid easily. It would be desirable to have a composition comprising DHA EE which is more stable.

### DETAILED DESCRIPTION OF THE INVENTION

It has been found, in accordance with this invention that a composition comprising the combination of DHA EE and Vitamin E Acetate (“Vit E Ac”) exhibits remarkable oxidative stability. The composition can be used as a nutraceutical or pharmaceutical and has enhanced shelf life.

In a preferred embodiment of this invention, the DHA EE used to make the composition of this invention is a highly concentrated DHA EE, meaning that the DHA EE contains at least 92% DHA EE by weight, preferably at least 93% DHA EE by weight, and more preferably at least 94% DHA EE by weight. In some embodiments, it is at least 95% DHA EE. Highly concentrated DHA EE is available from DSM Nutritional Products, Switzerland.

Vit E Ac can be obtained from DSM Nutritional Products, Switzerland.

### Definitions

As used throughout the specification and claims, the following definitions apply:

“Alpha tocopherol” means Vitamin E, any isomer

“Tocopherol acetate” means Vitamin E acetate.

“Vitamin E Acetate or Vit E Ac” means all racemic alpha-tocopherol acetate.

“Sole active ingredients” means that the composition may contain ingredients other than Vit E Ac and DHA, such as up to 0.25% (by weight) mixed natural tocopherols, and/or acetyl palmitate or other long chain fatty acid esters or the like which are not believed to substantially contribute to the bioactivity of the DHA EE and Vit E Ac composition. Other ingredients in the composition which may be present are processing aids such as fillers, binders, and other excipients.

“Highly concentrated DHA EE” refers to DHA EE containing at least 92% DHA EE.

### DESCRIPTION OF THE FIGURES

FIG. 1 is a graph showing the oxidative stability of highly concentrated DHA-EE with alpha-tocopherol and tocopherol acetate as described in Example 1.

FIG. 2 is a graph showing the variation of PV with alpha tocopherol and tocopherol acetate as described in Example 1.

FIG. 3 shows the variation of p-AV with alpha tocopherol and tocopherol acetate as described in Example 1.

FIG. 4 shows the variation of CD with alpha tocopherol ad tocopherol acetate as described in Example 1.

FIG. 5 shows the variation of DHA-EE levels with time as described in Example 1.

### RATIOS

The ratio of DHA EE to Vit E Ac can range from 10:1 to 1:10, based on weight % of the two ingredients. In some preferred embodiments, the ratio is 5:1 to 1:5; in another is it less than 2:1.

Dosages

In a preferred embodiment, the daily dose of Vit E Ac for an adult ranges from 500 IU up to 2000 IU. In a preferred embodiment, the amount of Vit E Ac present will range from 8000 IU to 1200 IU per daily dose. In some particularly preferred embodiments, the Vit E actetate is 1000 IU per daily dose.

The amount of DHA EE can range from 500 mg to 3 grams per daily dose. In a preferred embodiment, the DHA EE will range from 1.5 grams to 2.5 grams per daily dose. In some particularly preferred embodiments, the amount of DHA EE is 2 grams.

Thus, some particularly preferred daily doses, the invention comprise:

1000 IU Vit E Ac and 2 grams DHA EE at per daily dose;
800-1200 IU Vit E Ac and 1.5-2.5 g DHA EE per daily dose;
800 IU Vlt E Ac and 1.5 g DHA EE per daily dose; and
1200 IU Vit E and 2.5 g DHA EE per daily dose.

As these amounts are relatively large, it is preferred that an individual capsule or other oral dosage form contain a portion of the daily dosage. Thus the above daily dosages can preferably be administered, e.g. in two forms, each containing one-half of the daily dosages, or alternately three forms each containing one-third of the daily dosage, or so on. This is for the convenience of the patient and enhance compliance.

In some embodiments, the Vit E Acetate and DHA EE are combined with further known active ingredients which are conventionally administered to treat a condition.

In some embodiments, ascorbyl palmitate (AP) is also added to further improve oxidative stability of the Vit E Acetate and DHA EE combo. In some embodiments, the sole active ingredients are Vit E Acetate, DHA EE and AP.

In another embodiment of this invention, the composition comprises Vit E Acetate and DHA EE as its sole active ingredients. The sole active ingredients make up at least 95% by weight, preferably at least 96% by weight and more preferably at least 96.4% by weight of the final finished form (tablet, capsule, or the like). The additional ingredients are present as processing aids.

The composition according to the present invention comprising Vit E Ac and DHA EE may be used as nutraceutical compositions, i.e. as supplement to dietary compositions, i.e., (fortified) food/feed or beverages, or as compositions in dosage unit form such as pharmaceutical compositions, e.g., capsules, tablets, granules, pastes or effervescent formulations which may further comprise pharmaceutically acceptable carriers, excipients or diluents, including, but not limited to, lubricants, colorants, wetting agents, fillers, disintegrants and flavorants. The pastes may be filled into hard or soft gelatin capsules.

In preferred embodiments, the composition is in liquid form and is used to fill capsules, including soft get capsules.

The following non-limiting Examples further illustrate this invention.

### EXAMPLES

### Example 1

### Oxidative Stability of DHA-α-tocopherol and DHA-α-tocopherol acetate

Objective:

To study the effect of Vitamin E and Vitamin E acetate on the oxidative stability of 95% DHA concentrate EE.

Materials and Methods

Authentic standard Vitamin E (α-tocopherol) with the concentration of 1000 IU/g and Vitamin E acetate (α-tocopherol acetate) with the concentration of 1360 IU/g were purchased from Sigma-Aldrich, Canada. Fish oil concentrate with 95% docosahexaenoic acid (DHA), in the form of ethyl esters (95DHA-EE) which was already stabilized with 2 mg/g mixed natural tocopherols (MNT), was used in this study. Two basic methods were used to evaluate the oxidative stability.
1. Oxidative Stability Index or Oil Stability Index (OSI)
2. Conventional storage stability study

Oxidative Stability Index or Oil Stability Index

OSI of oil samples with different types and amounts of antioxidants were determined using Oxidative Stability Instrument. Vitamin E (α-tocopherol) and Vitamin E acetate (α-tocopherol acetate) were weighed into OSI tubes in 400 IU quantities and oil samples were added up to 5 g separately, with or without 0.5 mg/g ascorbyl palmitate (AP), in duplicate. Blank oil samples which contained about 2 mg/g MNT were used as the negative control to compare the efficacy of α-tocopherol and α-tocopherol acetate with and without ascorbyl palmitate. These samples were incubated in the Oxidative Stability Instrument at 70° C. while bubbling air through the oil with the air pressure of about 5.5 psi. The induction time of each oil sample was considered as the Oxidative Stability Index.

Conventional Storage Stability Study

For the storage stability study, 25 g samples of 95DHA-EE were prepared in glass amber bottles. α-tocopherol and α-tocopherol acetate were added with the concentration of 800 IU for each 2 g of DHA. Oil samples were prepared according to Table (1) to obtain the right combinations.

TABLE 1
Preparation of 25 g of sample blends

| | | Antioxidant | Amount of AOX for | |
|---|---|---|---|---|
| Sample | | (AOX) | 25 g oil (g) | Oil (g) |
| 1. | Blank | None | 0.05 | 25 |
| | (with 2 mg/g MNT) | | | |
| 2. | 95EE + α - | α -tocopherol | 9.501 | 15.499 |
| | tocopherol | | | |
| 3. | 95EE + α - | α -tocopherol | 9.501 | 15.487 |
| | tocopherol and AP | AP | 0.0125 | |
| 4. | 95EE + tocopherol | tocopherol | 6.986 | 18.014 |
| | acetate | acetate | | |
| 5. | 95EE with | tocopherol | 6.986 | 18.002 |
| | tocopherol | acetate | 0.0125 | |
| | acetate + AP | AP | | |
| 6. | 95EE + AP | AP | 0.0125 | 24.988 |

These oil samples were stored at the ambient temperature (23-25° C.) open to air, and the samples were taken at different times for the determination of peroxide value (PV), p-anisidine value, conjugated dienes (CD) and DHA contents in ethyl ester form.

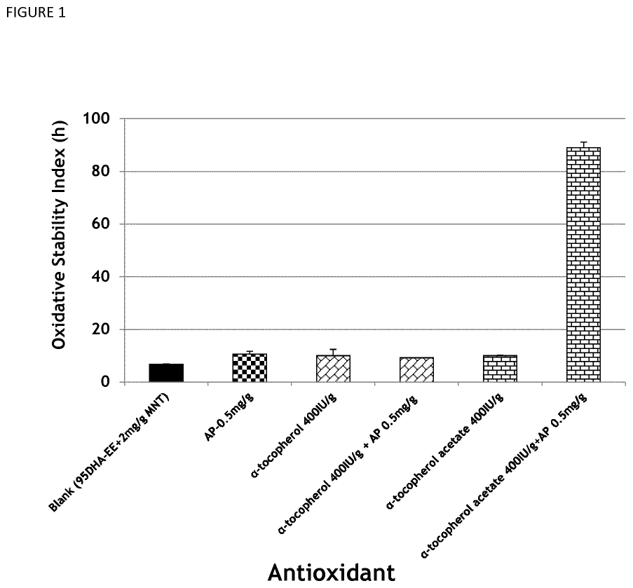

Results are given in FIGS. 1-5. Although ascorbyl palmitate, α-tocopherol and α-tocopherol acetate are known antioxidants, these compounds, when added each individually, did not improve the Oil Stability Index (FIG. 1) of DHA-EE which was already stabilized with 2 mg/g mixed natural tocopherols (MNT). Even the addition of ascorbyl palmitate, which is commonly used as an antioxidant in fish oil, did not improve the antioxidant activity of α-tocopherol as determined by the OSI, whereas ascorbyl palmitate considerably improved the Oil Stability Index when combined with α-tocopherol acetate.

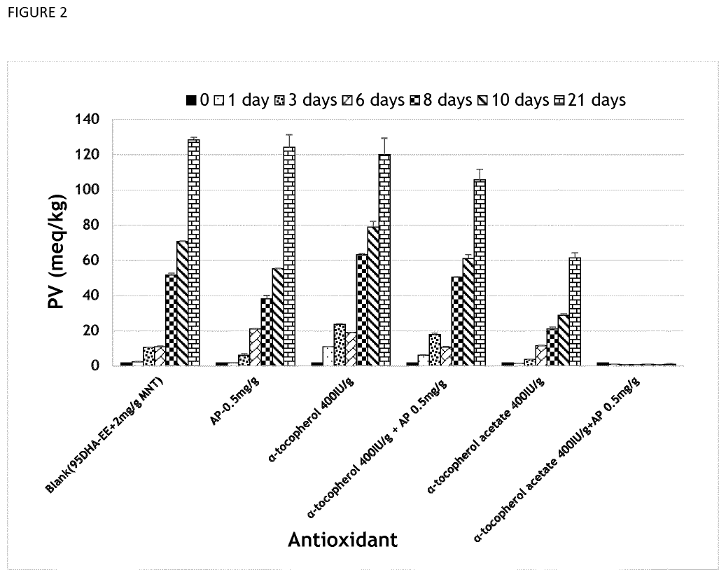

Hydroperoxides are the primary oxidation products which indicate the level of initial oxidation of unsaturated fatty acids and, these products are determined as the peroxide value (PV). PV of all samples increased considerably except the oil samples containing ascorbyl palmitate+α-tocopherol acetate indicating the strong synergistic antioxidant activity of these 2 compounds together (FIG. 2).

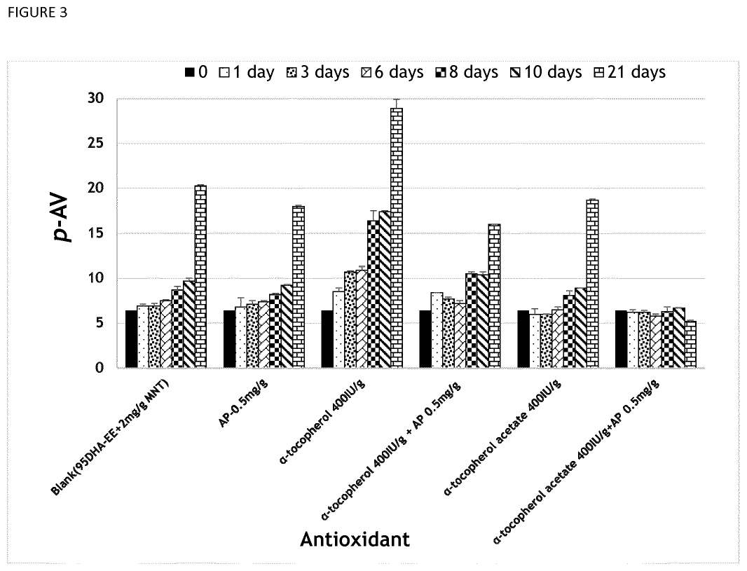

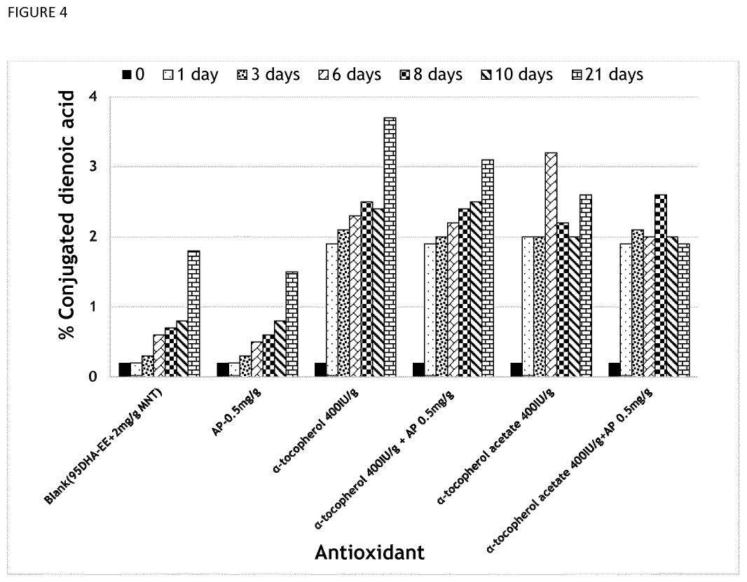

Secondary oxidation products which are determined as anisidine reactive substances are determined as the p-AV (FIG. 3). There was no increase of p-AV in samples containing ascorbyl palmitate plus α-tocopherol acetate at all during the storage whereas this value of all other samples gradually increased during the storage period of 21 days. Conjugated dienes which also indicate the level primary oxidation did not increase in the samples containing ascorbyl palmitate plus α-tocopherol acetate (FIG. 4). Thus, all these stability indices clearly demonstrate that addition of ascorbyl palmitate considerably improve the oxidative stability of to the a tocopherol acetate and DHA-EE combo.

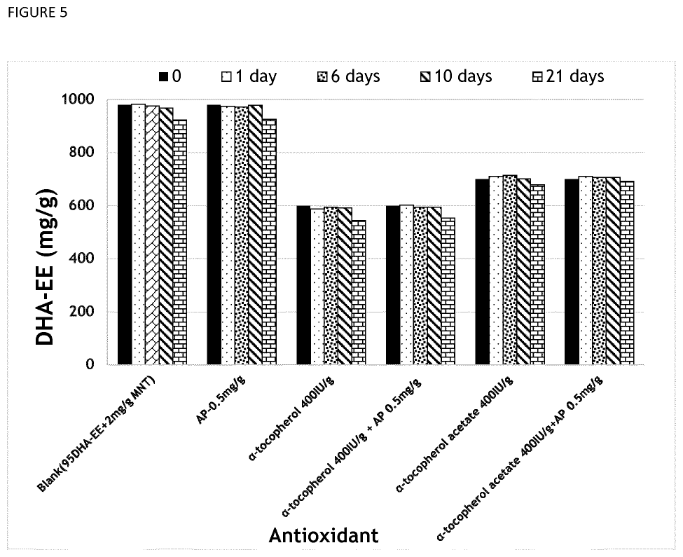

In addition, there was no considerable variation in the amount of DHA in the oil samples after 21 days of storage at ambient temperature (FIG. 5).

A soft gel capsule contains (by weight percent):

| | |
|---|---|
| DHA-EE | 68.2 |
| Vit E Ac | 27.9 |
| Mixed natural tocopherols | 0.2 |
| AP | 0.05 |
| Other long chain fatty acids | ~3.7 |

1. A composition comprising Docosapentaenoic acid ethyl ester (DHA EE) and Vitamin E Acetate (Vit E Ac).

2. A composition according to claim 1 where DHA EE and VIt E Acetate are the sole active ingredients.

3. A composition according to claim 1 where the DHA EE is a highly concentrated DHA EE.

4. A composition according to claim 3 where the DHA EE is at least 90% DHA EE.

5. A composition according to claim 1 where DHA EE, Vit E Ac and ascorbyl palmitate as the sole active ingredients.

6. A composition according to claim 5 where the DHA EE is a highly concentrated DHA EE.

7. A softgel capsule comprising a composition according to claim 1.
